(19) United States
(12) Patent Application Publication
(10) Pub. No.: US 2025/0281581 A1
(43) Pub. Date: Sep. 11, 2025

(54) METHODS OF CANCER TREATMENT BY INTRATUMORAL INJECTION OF MUTANT COLLAGENASE

(71) Applicant: REJUVEN DERMACEUTICAL CO., LTD., HANGZHOU (CN)

(72) Inventors: CANG YONG, HANGZHOU (CN); YIHUI LIU, HANGZHOU (CN); SHENG SHI, HANGZHOU (CN)

(73) Assignee: REJUVEN DERMACEUTICAL CO., LTD., HANGZHOU, ZJ (CN)

(21) Appl. No.: 18/259,576

(22) PCT Filed: Dec. 9, 2021
(86) PCT No.: PCT/CN2021/136921

§ 371 Date: Jun. 27, 2023

(30) Foreign Application Priority Data
Dec. 29, 2020 (CN) 202011596224.5

(51) Int. Cl. A61K 38/48; A61P 35/00

(52) U.S. Cl. CPC A61K 38/4886; A61P 35/00; C12Y 304/24

(57) ABSTRACT

The disclosed matter relates to methods of cancer treatment by intratumoral injection of mutant collagenase. The disclosed matter provides methods of cancer treatment, wherein injecting a composition comprising mutant collagenase into a tumor of an individual with cancer to reduce the abundance of collagen in the extracellular matrix of the tumor microenvironment. Applying the methods of the disclosed matter can lead to reduction of the stiffness and volume of the treated tumor and can induce the infiltration of immune cells, especially cytotoxic T lymphocytes (CTL). When combined with immune checkpoint blockade therapy, targeted therapy and chemotherapy, the mutant collagenase injection provided by the present invention exhibits synergistic antitumor activity.

### CROSS REFERENCE TO RELATED APPLICATIONS

This U.S. non provisional application claims priority to International Patent Application No. PCT/CN2021/136921, which was filed on Dec. 9, 2021, which is related to, and claims priority from Chinese Patent Application No. 202011596224.5, entitled “Methods of cancer treatment by intratumoral injection of mutant collagenase”, which was filed on Dec. 29, 2020, the entire contents of which is incorporated herein by reference in its entirety (including all appendices thereto).

### SEQUENCE LISING INFORMATION

The instant application contains a Sequence Listing which has been filed electronically in ASCII format and is hereby incorporated by reference in its entirety. Said ASCII copy, created on May 31, 2023, is named US_HXPCT2103-seql.txt and is 12,372 bytes in size.

### FIELD

The present disclosure relates to the application of biopharmaceuticals for the methods of cancer treatment, and specifically relates to methods of cancer treatment by intratumoral injection of mutant collagenase.

### BACKGROUND

Cancer is one of the most intractable problems on public health in the world today. In 2018, there were approximately 18 million cancer patients and 9.6 million people died of cancer, that is, one in every six people died of cancer [1,2]. The World Health Organization predicts that by 2040, there will be 29.4 million cancer patients worldwide^{[1]}. In order to treat cancer, developing an effective cancer therapy has become an important task. Diseases characterized by tumor fibrosis, such as melanoma, pancreatic cancer and cholangiocarcinoma, are noteworthy, because fibrosis of the tumor microenvironment making it more difficult for immune cells or medicaments to act. In 2020, there are approximately 320,000 melanoma patients and 500,000 pancreatic cancer patients worldwide, and pancreatic cancer has also become the cancer with the seventh highest number of deaths, with approximately 470,000 people died^{[3, 4]}. Today, the methods of treating cancer are mainly divided into: surgical resection, radiation therapy, anti-cancer drug therapy (chemotherapy), and combination therapy with immune blockade inhibitor. However, based on the characteristics of tumors, it has become a difficult problem for targeted drugs to reach tumor cells effectively and exert effects. The extracellular matrix of tumors, such as collagen and hyaluronic acid, promotes tumor growth and metastasis, acts as a natural drug diffusion barrier, and blocks the infiltration of lymphocytes and targeted drugs^{[5, 6]}. For example, collagen is involved in the fibrosis of cancer tissue, and in areas where collagen being abundant, fibronectin, hyaluronic acid, laminin and matrix metalloproteinases interacting with collagen to affect cancer cells. As another example, hyaluronic acid is a glycosaminoglycan which accumulates on the surface of tumor cells and in the space around them. As immune cells moving toward the tumor, hyaluronic acid forms a barrier, compressing blood vessels and hindering chemotherapy. Therefore, for fibrotic and hard tumors, current anti-tumor therapies could not work well. In recent years, people have been actively looking for pathways that can assist the treatment.

The structures of solid tumors are complex, containing not only tumor cells, but also vasculature, extracellular matrix, stromal cells, and immune cells. Among them, the tumor microenvironment accounts for a larger part of the tumor mass, the extracellular matrix is the most abundant component in the tumor microenvironment, and in extracellular matrix, collagen and hyaluronic acid are the most abundant two^{[6]}. Some studies have found that the changes of extracellular matrix and stromal components occurred in tumor microenvironment are factors that may affect the treatment effects and cause tissue stiffening. It is also proposed that the tumor hardness is positively correlated with the volume of the tumor rich in collagen and fibroblasts. Further analysis also found the density of collagen is positively correlated with tumor hardness, while hyaluronic acid density is not significantly correlated with tumor hardness^{[6, 7]}. In solid tumors, the role of the extracellular matrix differs from that in normal organs, and intratumoral signaling, transport mechanisms, metabolism, oxygenation, and immunogenicity are all affected by the extracellular matrix. Some studies have also proposed that collagen does not only constitute the scaffold of the tumor microenvironment, but also affects the tumor microenvironment and participates in the process of tumor progression^{[8]}.

An ideal anti-cancer method is to kill cancer cells without harming normal cells. Current chemotherapeutic drugs have not solved this problem very well. Therefore, there are a series of targeted therapy which exploit tumor specific immune activity and immunotherapy approaches. Among them, the immunotherapy method with Anti-Programme Death-1 (PD-1)/PD-1 ligand (PD-L1) monoclonal antibodies targeting the immune checkpoint has developed rapidly in recent years. When combined with current treatment means, this immunotherapy method showed good efficacy in treating various tumors or advanced cancers such as melanoma, lung cancer, kidney cancer and etc.^{[9-11]}. However, studies have found that 40%-60% of melanoma patients have resistance to immune checkpoint blockade therapy (such as PD-1/PD-L1 blockade, CTLA4 inhibition), and another part of the patients relapsed within two years^{[12]}. In the past two years, there are also studies showing the limitation of the efficacy of PD-1/PD-L1 immunotherapy on solid tumors, and only some patients had clinical response to PD-1/PD-L1 therapy, while the therapy

is ineffective for a large number of patients [13]. There are studies showing that the resistance mechanism to PD-1//PD-L1 may γ) signaling, and immune

microenvironment heterogeneity and the like^{[13]}. In 2018, there is a review suggesting that mutant collagenase can adjust the tumor microenvironment and improve the efficiency of drug delivery by inhibiting the collagen synthesis pathway or using mutant collagenase to deplete tumor collagen. In theory, reducing the extracellular matrix of tumors can increase drug penetration. This leads to the increase of drug concentration in tumor cells^{[8]}. However, drug Xiaflex® in the prior art is used to treat Dupuytren's Contracture with lysis of collagen, and the application of mutant collagenase for cancer treatment has not been established^{[8]}.

### SUMMARY OF INVENTION

### Problems to be Solved by the Invention

In view of the problems existing in the prior art, the present disclosure provides methods of cancer treatment by intratumoral injection of mutant collagenase. The mutant collagenase is used for lysis of collagen in the extracellular matrix, to regulate the intra-tumoral cell microenvironment, and current anti-cancer treatment means (such as chemotherapy, immunotherapy, targeted therapy) and the like are combined for the purpose of improving the anti-cancer effect.

### Means for Solving the Problems

In view of the above-mentioned problems existing in the prior art, the present inventors have conducted in-depth research and through trial and error. Based on the fact that several types of tumors, including pancreatic cancer and cholangiocarcinoma, characterized in the dense collagen fibers in the tumor microenvironment, the present disclosure provides a method of cancer treatment by intratumoral injection of mutant collagenase. Specifically, the present disclosure reduces the abundance of collagen in the extracellular matrix of the tumor microenvironment via injecting recombinant mutant collagenase purified from Clostridium spp. (specifically, Clostridium histolyticum) into solid tumors. Thus, the present disclosure has been completed.

The present disclosure is intended to the lysis of the collagen in the solid tumor microenvironment through mutant collagenase for improving the diffusion and penetration of drugs in the methods of treatment of tumor. In the present disclosure, mutant collagenase is directly injected into the intratumoral microenvironment to take effects; and at the same time, it is used in combination with targeted therapy and immunotherapy method to provide more space for infiltration of immune cells and targeted drug's diffusion and penetration. The mechanism of action of injecting mutant collagenase is shown in FIG. 1.

In a first aspect of the present disclosure, a method of cancer treatment is provided, wherein injecting a composition comprising mutant collagenase into a tumor of an individual with cancer to reduce the abundance of collagen in the extracellular matrix of the tumor microenvironment.

In a specific embodiment, said tumor is a solid tumor; preferably, said tumor is any one selected from the group consisting of melanoma, pancreatic cancer, cholangiocarcinoma, breast cancer, colorectal cancer, ovarian cancer, and lung cancer; more preferably, said tumor is any one selected from the group consisting of melanoma, pancreatic cancer and cholangiocarcinoma.

In some other specific embodiments, the mutant collagenase in said composition is recombinant mutant collagenase with purity of more than 98%; preferably, said recombinant mutant collagenase is mutant collagenase expressed by Clostridium histolyticum with 451 site of mutant collagenase ColH being mutated into aspartic acid, and the amino acid sequence of said recombinant mutant collagenase is given by SEQ ID NO: 2.

More specifically, said injecting is multi-point injection or two-point injection, and the concentration of said mutant collagenase in said composition is 0.005-0.15 mg/100 μl.

Also, the method of cancer treatment provided in the first aspect of the present disclosure can be applied in combination with chemotherapy, immunotherapy or targeted therapy.

In a second aspect of the present disclosure, a use of a composition comprising mutant collagenase in the manufacture of a medicament for the treatment of cancer is provided.

In a specific embodiment, said cancer is malignant tumor; preferably, said cancer is any one selected from the group consisting of melanoma, pancreatic cancer, cholangiocarcinoma, breast cancer, colorectal cancer, ovarian cancer, and lung cancer; more preferably, said cancer is any one selected from the group consisting of melanoma, pancreatic cancer and cholangiocarcinoma.

0 In some other specific embodiments, the mutant collagenase in said composition is recombinant mutant collagenase with purity of more than 98%; preferably, said recombinant mutant collagenase is mutant collagenase expressed byClostridium histolyticum with 451 site of mutant collagenase ColH being mutated into aspartic acid, and the amino acid sequence of said recombinant mutant collagenase is given by SEQ ID NO: 2.

More specifically, said composition further comprises a pharmaceutically acceptable carrier.

Also, the dosage form of the composition provided in the second aspect of the present disclosure is injection; perfectly, preferably, said injection is injection solution or powder-injection.

### BRIEF DESCRIPTION OF THE DRAWINGS

FIG. 1 is an illustration showing the mechanism of action of injecting mutant collagenase.

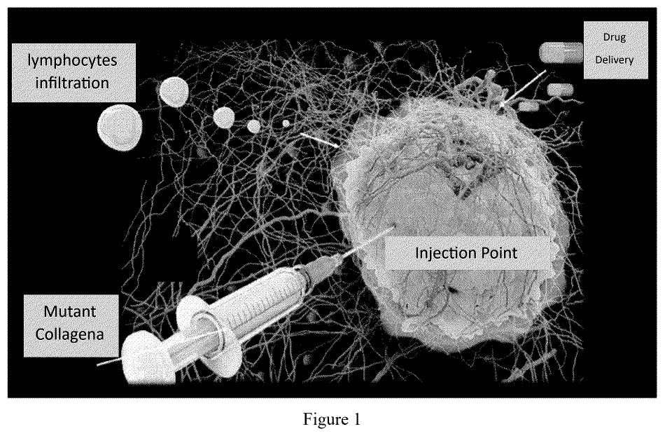

FIG. 2 is an illustration showing the tumor volume of B16F10 melanoma mouse model over time.

FIG. 3 is an illustration showing the distribution of collagen, observed with Masson's trichrome stain, in tumor tissues with different volumes derived from B16F10 melanoma mouse model.

FIG. 4 is an illustration showing the change of the tumor tissues of B16F10

melanoma mouse model, after the injection of 0.15 mg/100 μl mutant collagenase, wherein PBS is phosphate buffered saline.

FIG. 5 is an illustration showing the change of the tumor tissues of B16F10 melanoma mouse model, after the injection of 0.05 mg/200 μl mutant collagenase.

FIG. 6 is an illustration showing the distribution of collagen in tumor tissue of B16F10 melanoma mouse model after the injection of phosphate buffered saline (PBS) and 0.15 mg/100 μl mutant collagenase.

FIG. 7 is a schematic diagram of multi-point injection in the mouse.

FIG. 8 is a schematic diagram of subcutaneous injection in the mouse.

FIG. 9 is an illustration showing the change of tumor tissues of B16F10

melanoma mouse model after multi-point injection of different low-dose mutant collagenase.

FIG. 10 is an illustration showing the change after multi-point injection of mutant collagenase and phosphate buffered saline (PBS) in the tumor tissues of B16F10 melanoma mouse model.

FIG. 11 is an illustration showing the distribution of collagen observed by H&E staining, after multi-point injection of 0.05 mg/200 μl in B16F10 melanoma mouse model.

FIG. 12 is an illustration showing the tumor morphology change after multi-point injection of 0.05 mg/200 μl in B16F10 melanoma mouse model.

FIG. 13 is an illustration showing the distribution of collagen, observed with Masson's trichrome stain, in pancreatic cancer tissues of PAN02 pancreatic cancer mouse model.

FIG. 14 is an illustration showing the change after two-point injection of phosphate buffered saline (PBS) and 0.025 mg/100 μl mutant collagenase in the tumor tissues of PAN02 pancreatic cancer mouse model.

FIG. 15 is an illustration showing the distribution of collagen, observed with Masson's trichrome stain, after two-point injection of phosphate buffered saline (PBS) and 0.025 mg/100 μl mutant collagenase in the tumor tissues of PAN02 pancreatic cancer mouse model.

FIG. 16 is an illustration showing the distribution of collagen observed by H&E staining, after two-point injection of 0.025 mg/100 μl mutant collagenase in the tumor tissues of PAN02 pancreatic cancer mouse model (with specific reference to FIG. 17).

FIG. 17 is an illustration showing infiltration of immune cells after two-point injection of 0.025 mg/100 μl in PAN02 pancreatic cancer mouse model.

FIG. 18 is an illustration showing the change of tumor size and volume after injection of mutant collagenase in PAN02 pancreatic cancer mouse model.

FIG. 19 is an illustration showing the trend of tumor volume change after injection of mutant collagenase in PAN02 pancreatic cancer mouse model.

### DETAILED DESCRIPTIONS OF INVENTION

### Definition

Unless specifically defined herein, all technical and scientific terms used herein generally have the same meaning as commonly understood by one of ordinary skill in the art to which this invention belongs. For purposes of the present disclosure, the following terms are defined below.

In specific embodiments of the present disclosure, said “mutated collagenolytic protease” preferably is the recombinant mutant collagenase mentioned in Chinese Patent CN108949730A, which is expressed by Clostridium histolyticum with 451 site of mutant collagenase ColH being mutated into aspartic acid, and the amino acid sequence of said recombinant mutant collagenase is given by SEQ ID NO: 2,and the encoding sequence of the amino acid sequence is given by SEQ ID NO: 1.Said mutant collagenase ColH is numbered as RJV001. For a detailed manufacturing method of the mutant collagenase, refer to Chinese Patent CN108949730A.

In the present disclosure, “mutated collagenolytic protease” may also be referred to as “mutant collagenase”, both of which are interchangeable and have the same meaning.

The term “cancer” refers to a broad category of disorders characterized by hyperproliferative cell growth in vitro (e.g., transformed cells) or in vivo. Specific examples of cancer include, but are not limited to, blood cancer, colon cancer, rectal cancer, renal cell carcinoma, liver cancer, non-small cell carcinoma of the lung, small intestine cancer, esophageal cancer, melanoma, bone cancer, pancreatic cancer, skin cancer, head and neck cancer, skin or intraocular melanoma, uterine cancer, ovarian cancer, rectal cancer, anal cancer, stomach cancer, testicular cancer, uterine cancer, fallopian tube cancer, endometrial cancer, cervical cancer, vaginal cancer, vulva cancer, Hodgkin's disease, Lymphoma, Non-Hodgkin's, endocrine system cancer, thyroid cancer, parathyroid cancer, adrenal cancer, soft tissue sarcoma, urethral cancer, penile cancer, childhood solid tumors, bladder cancer, kidney or ureteral cancer, renal pelvis cancer, central nervous system (CNS) tumor, primary CNS lymphoma, tumor angiogenesis, spinal tumor, brain stem glioma, pituitary adenoma, Kaposi's sarcoma, epidermoid carcinoma, squamous cell carcinoma, T-cell lymphoma, environmentally induced cancer, a combination of said cancers and metastatic lesions of said cancers.

The terms “treat”, “treatment” and “treating” refer to the reduction or amelioration of progression, severity and/or duration of a proliferative disorder, or the amelioration of one or more symptoms (preferably, one or more discernible symptoms) of a proliferative disorder resulting from the administration of one or more therapies. In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating” refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count, or prolonging survival of an individual.

The term “treatment of cancer” or “cancer treatment” is not intended to be an absolute term. In some aspects, the methods of the invention seek to reduce the size of a tumor or number of cancer cells, cause a cancer to go into remission, or prevent growth in size or cell number of cancer cells. In some circumstances, treatment with the leads to an improved prognosis.

The term “multi-point injection” in the present disclosure refers to orthotopic mutant collagenase per point.

The term “two-point injection” in the present disclosure refers to orthotopic injections of 2 points into tumor tissue, subcutaneously in mice, with 50 μl of mutant collagenase per point.

In the present specification, unless otherwise specified, “%” refers to percentage by mass.

In the present disclosure, the term “pharmaceutically acceptable carrier” refers to auxiliary ingredients suitable for being compatible with cells, tissues or organs of the human or animal body, and do not induce toxic side effects such as toxicity, irritation, allergy, etc. Exemplary pharmaceutically acceptable carriers are well known in the art and include solvents, solubilizers, cosolvent, emulsifiers, taste corrigent, smell corrigent, colorants, binders, disintegrants, fillers, lubricants, wetting agents, osmotic pressure regulators, pH regulators, stabilizers, surfactants and/or preservatives.

In the present disclosure, the term “effective amount” refers to an amount of a compound which is sufficient to provide a desired effect but with no toxicity or with acceptable toxicity. The amount may vary from subject to subject, depending on the species, age, and physical condition of the subject, the severity of the disease that is being treated, the particular compound used, its mode of administration, and etc. A suitable effective amount may be determined by one of ordinary skill in the art.

In the present disclosure, the term “therapeutically effective amount” is used to indicate an amount of active compound or pharmaceutical agent, that elicits the biological or medicinal response indicated. This response may occur in a tissue, system (animals including human) that is being sought by a researcher, veterinarian, medical doctor or other clinician.

### Invention Effects

As can be seen from the technical solution of the present disclosure, compared with the prior art, the technical solution of the present disclosure has the following beneficial effects:
1. In the present disclosure, recombinant mutant collagenase is injected into a tumor, which can lead to lysis of collagen in the tumor, changing extracellular matrix in the microenvironment, so as to enable the effective invasion of the killer immune cells (mainly T cells) and targeted drugs. The methods provided in the present disclosure can lead to reduction of the stiffness and volume of the treated tumor and can induce the infiltration of immune cells, especially cytotoxic T lymphocytes (CTL). In particular, through the methods provided in the present disclosure, the fibrosing of tumor cells can be reduced which means reducing abundance of collagen in the extracellular matrix, thereby softening the tumor and reducing its hardness.
2. The indications for the treatment methods provided by the present disclosure are solid tumor and fibrous tumor-associated cancer, and the present disclosure especially has therapeutic effects on fibrous tumors such as melanoma, pancreatic cancer, cholangiocarcinoma and the like. The treatment methods provided by the present disclosure also have good treatment effects on other cancers having dense extracellular matrix characteristic (such as breast cancer, colorectal cancer, ovarian cancer, and lung cancer).
3. The mutant collagenase injection provided by the present disclosure can be directly injected into tumor cells/cancer cells with multi-point. When combined with the immune checkpoint blockade therapy, targeted therapy and chemotherapy, the mutant collagenase injection provided by the present disclosure exhibits synergistic antitumor activity and achieves the antitumor treatment purpose.

In order to make the aforementioned and other objectives, features and advantages of the present disclosure comprehensible, preferred examples accompanied with figures are described in detail below.

### EXAMPLES

The specific examples illustrated in the present disclosure are merely examples of the present disclosure, and the present disclosure is not limited to the specific examples described below. For those skilled in the art, any equivalent modifications and substitutions to the examples described below also fall within the scope of the present disclosure. Therefore, all equivalent changes and modifications made without departing from the spirit and scope of the present disclosure should be encompassed in the scope of the present disclosure.

It is to be understood that the embodiments of the present disclosure which have been described are merely illustrative of some of the applications of the principles of the present disclosure. Numerous modifications may be made by those skilled in the art based upon the teachings presented herein without departing from the true spirit and scope of the invention. The contents of all references, patents and published patent applications cited throughout this application are hereby incorporated by reference in their entirety for all purposes.

If the specific conditions are not indicated in the examples, the conventional conditions, for example, a condition as described in MOLECULAR CLONING: A LABORATORY MANUAL THIRD EDITION (authored by J. Sambrook et al., Science Press, 2002), or the conditions suggested by the manufacturer shall be followed. Any reagents or instruments used, without no statement of manufacture, are conventional products that can be obtained by market purchase. Various specific details are given in the following specific embodiments in order to better illustrate the present disclosure. It should be appreciated by a person skilled in the art that the invention may also be implemented without certain specific details. In some other examples, methods, means, apparatus and steps well known to a person skilled in the art are not described in detail, so as to highlight the essence of the invention.

Unless specifically defined herein, all technical and scientific terms used herein generally have the same meaning as commonly understood by one of ordinary skill in the art. Unless otherwise stated, all of the units used in the present disclosure are international standard units, and the numerical values or numeral ranges should be understood as including unavoidable industrial systematic errors.

### Example 1 Injection of Mutant Collagenase on the Basis of B16F10 Melanoma Mouse Model

### Instruments and Materials

(1) The mouse model was constructed by implanting homograft of mouse melanoma cells B16F10 into C57BL/6 mouse subcutaneously, and tumor tissues with the volumes of 100 mm³, 300 mm³, 600 mm³ and 1000 mm³ were selected for observation.
(2) Mouse tumor tissues with different sizes were selected to perform experiments using different dosages of mutant collagenase. The grouping for different dosages of mutant collagenase is as follows:

Dosage group A (0.15 mg/100 μl): the mice were numbered as 5-N, 5-L, 3-N and 5-LL respectively; corresponding to tumor tissue volumes of 300 mm³, 394 mm³, 467mm³ and 1139 mm³ respectively, wherein the mouse numbered 5-N was injected with 100 μl of 0.9% phosphate buffered saline (PBS), and each of the mice numbered as 5-L, 3-N and 5-LL was injected with 0.15 mg/100 μl of mutant collagenase respectively;

Dosage group B (0.05 mg/200 μl): the mice were numbered as 4-R, 4-RR and 5-RR respectively; corresponding to tumor tissue volumes of 446 mm³, 487 mm³ and 1142mm³, respectively.

### Experimental Methods

(1) The distribution and content of collagen in tumor tissues with volumes of 100 mm³, 300 mm³, 600 mm³ and 1000 mm³ were observed with Masson's trichrome stain under magnifications of 5× and 10×.
(2) The mutant collagenase and PBS were injected, at one single point, into the tumor tissues of the mice with different numbers respectively, and the change of the tumor tissues were observed 24 hours later. The injection dosages of mutant collagenase in different groups of experiments were 0.15 mg/100 μl in dosage group A and 0.05 mg/200 μl in dosage group B, respectively.

### Experimental Results

(1) With the growth of the tumor, collagen showed an overall increasing trend; and

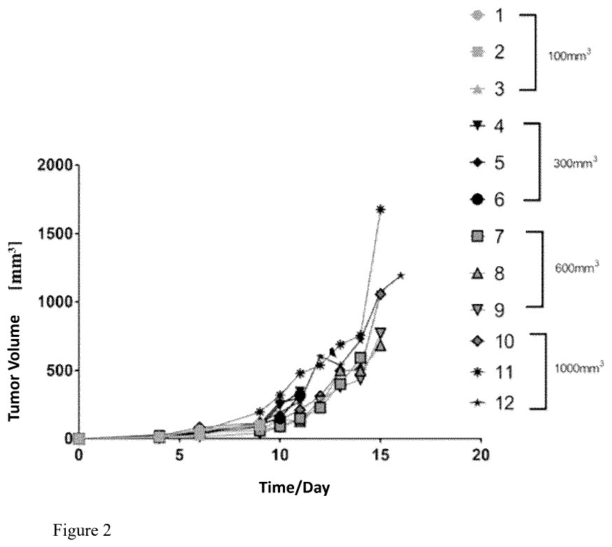

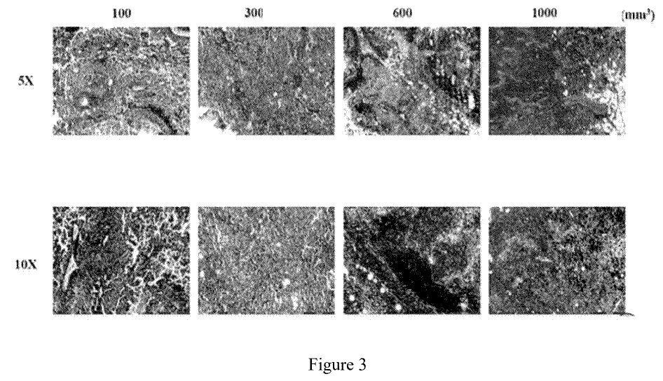

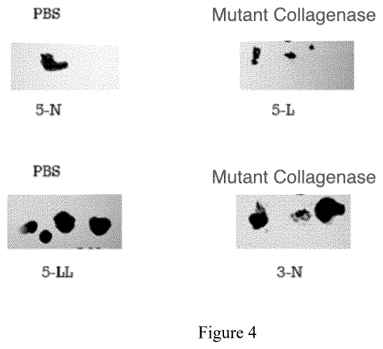

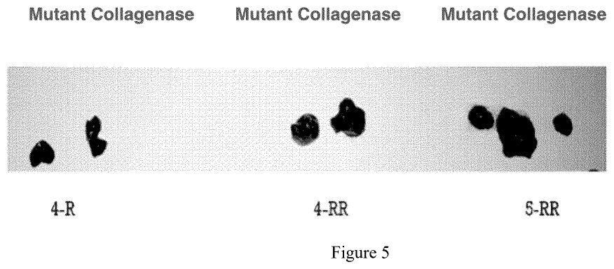

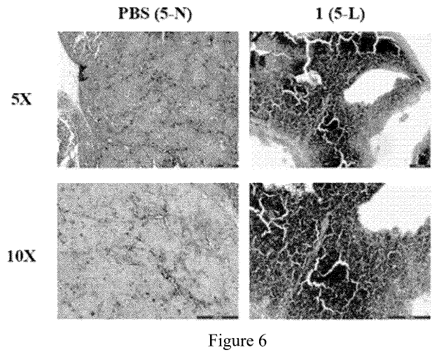

it could be clearly observed with the tumor tissue volume being 300 mm³, and it became abundant when the volume reached 600 mm³ (with specific reference to FIG. 2 and FIG. 3).
(2) Compared with the tumor tissue injected with PBS, the texture of the tumor tissue after the injection of mutant collagenase with different dosage showed a tendency of becoming smaller, softer and more dispersed (with specific reference to FIG. 4-FIG. 5).
(3) With Masson's trichrome stain, it can be observed that, in the tumor tissue (number 5-L) injected with mutant collagenase having a concentration of 0.15 mg/100 μl, a large amount of collagen was cleaved, disappeared; while the tumor tissue injected with PBS showed a large accumulation of collagen (with specific reference to FIG. 6).

### Example 2 Multi-Point Injection of Mutant Collagenase on the Basis of B16F10 Melanoma Mouse Model

### Instruments and Materials

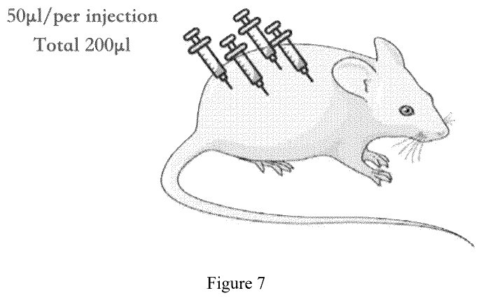

The mouse model is C57BL/6 mouse with implantation of mouse melanoma B16F10 homograft, tumor tissues with different volumes were selected to conduct multi-point injection (specifically, injection of 4 points, 50 μl per point) to perform the experiment (for detailed injection way, refer to FIG. 7). Details of the grouping and injection dosages are as follows:

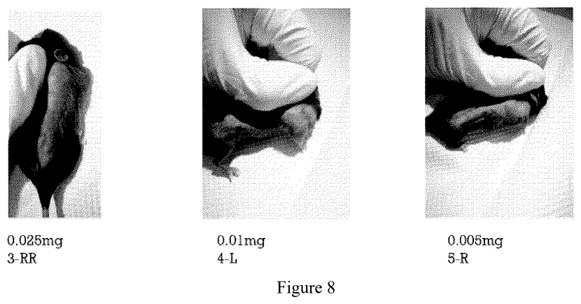

Group A: The mice were numbered as 3-RR, 4-L and 5-R respectively, corresponding to tumor tissue volumes of 1055 mm³, 449 mm³ and 423 mm³ respectively. For 3-RR, 4-L, and 5-R, the dosages of the mutant collagenase injected were 0.025 mg/200 μl, 0.01 mg/200 μl and 0.005 mg/200 μl respectively (with specific reference to FIG. 8);

Group B: The mice were numbered as 4-N and 3-R respectively, corresponding to tumor tissue volumes of 2690 mm³ and 2354 mm³ respectively. For 4-N, the dosage of the mutant collagenase injected was 0.05 mg/200 μl, and for 3-R the dosages of PBS injected was 0.05 mg/200 μl.

### Experimental Methods

For group A, three different formulations of low-dose mutant collagenase were injected into the tissues in the B15F10 melanoma mouse model and the change was observed; for group B, multi-point injections of mutant collagenase were conducted in tumor tissues with big volumes and the change was observed (in comparison with injection of PBS). The distribution of collagen in tumor tissues after the injection of mutant collagenase was observed with Masson's trichrome stain.

### Experimental Results

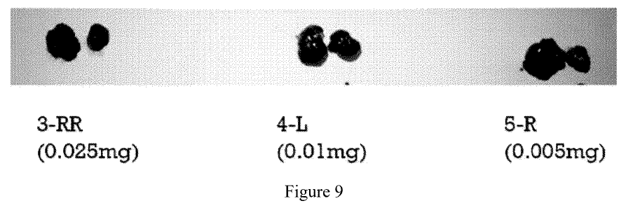

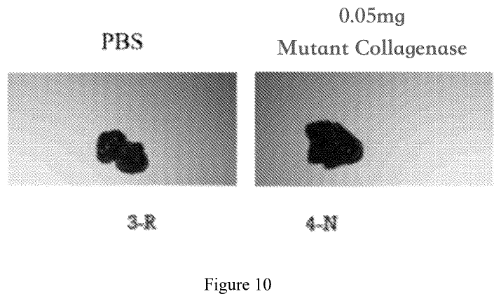

(1) In the early stage of the tumor growth of melanoma, multi-point injection of mutant collagenase also ameliorated the fibrosing of tumor tissue; and a tendency of tumor tissue becoming smaller, more dispersed, and softer was shown (with specific reference to FIG. 9).
(2) In tumor tissue with big volume, it was clearly observed that the injection of mutant collagenase caused the tumor tissue to disperse, and soften (with specific reference to FIG. 10).

### Example 3 Injection of Mutant Collagenase for B16F10 Melanoma Mouse Model

### Instruments and Materials

B16F10 melanoma cell line, C57BL/6 mouse with the age of 6-8 weeks.

H&E staining materials: fixative, hematoxylin staining solution, eosin staining solution, dilute hydrochloric acid ethanol solution, culture flask, culture dish, ophthalmic forceps, coverslip, slide, microscope.

Immunohistochemical/fluorescence analysis materials: sections, reagents/kits, instruments/consumables.

### Experimental Methods

(1) Model construction: Homograft of living cells of mouse melanoma cells B16F10 was implanted into C57BL/6 mouse subcutaneously. Volume testing and dissection analysis for different volumes were performed; tissue section H&E staining/collagen staining and immunohistochemical/fluorescence analysis were performed.
(2) Gradient administration of mutant collagenase and optimization of conditions: the inoculated model mouse was administrated; survival analysis and side effect analysis were performed; analysis of tumor volume, texture and metastasis was performed; tissue section H&E staining/collagen staining and immunohistochemical/fluorescence analysis were performed; flow cytometry analysis of tumor-infiltrating immune cells (TILS) was performed.

### Experimental Results

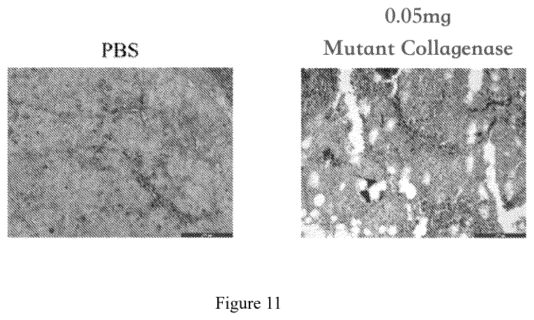

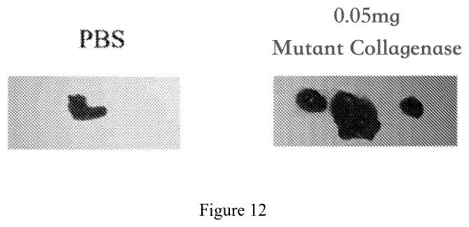

Injection of 4 points, 200 μl, 0.0125 mg/250 mm³.
(1) The tumor collagen decreased, and the abundance decreased (with specific reference to FIG. 11).
(2) The tumor became soft, and the tissue became loose (with specific reference to FIG. 12).

### Example 4 Two-Point Injection of Mutant Collagenase on the Basis of PAN02 Pancreatic Cancer Mouse Model

### Instruments and Materials

The mouse model was constructed by inoculating the mouse pancreatic cancer cell line PAN02 into C57BL/6 mouse subcutaneously; and tumor tissues with different volumes were selected to conduct two-point injection (specifically, injection of 2 points, 50 μl per point, in total comprising 0.025 mg mutant collagenase)

### Experimental Methods

(1) The distribution of collagen in pancreatic cancer tissues in PAN02 mouse model was observed with Masson's trichrome stain. The mice were numbered as 2-L, 1-R and 1-RR respectively.
(2) 0.025 mg of mutant collagenase was injected into the pancreatic cancer tissue in the mouse model of this example (the mouse number was 2-N) in the way of 2 points and the change was observed; this was compared with the group (mouse number was 2-RR) in which 100 μl of 0.9% phosphate buffered saline was injected. The distribution of collagen in tumor tissues after injection of mutant collagenase was observed with Masson's trichrome stain.

### Experimental Results

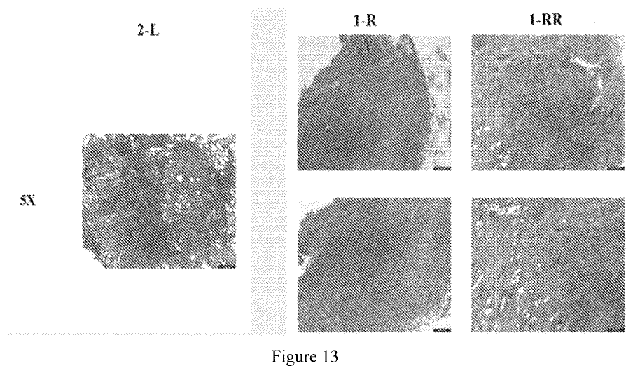

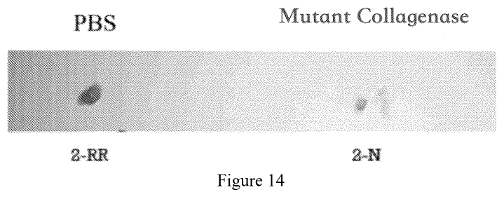

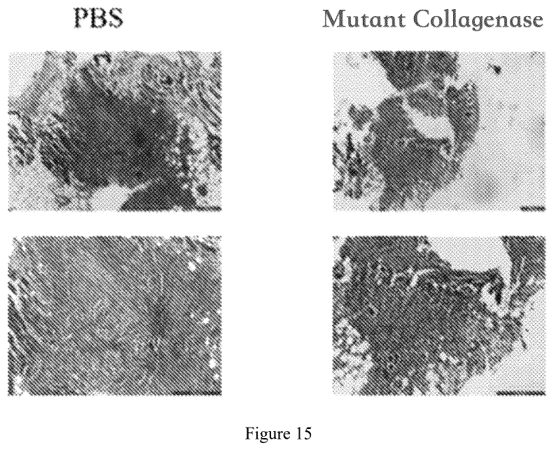

(1) With Masson's trichrome stain, a large amount of collagen was observed in the pancreatic cancer tissues (tissue volume less than 100 mm³) in PAN02 mouse model (with specific reference to FIG. 13).
(2) Significant diminishing was observed in the pancreatic cancer tissue of the mouse model, numbered as 2-N, injected with mutant collagenase (with specific reference to FIG. 14), and a large amount of collagen disappeared; the pancreatic cancer tissue of the mouse model, numbered as 2-RR, injected with PBS, still exhibited a large amount of collagen (with specific reference to FIG. 15).

### Example 5 Injection of Mutant Collagenase for PAN02 Pancreatic Ductal Adenocarcinoma Mouse Model

### Instruments and Materials

PAN02 pancreatic cancer tumor cell line, C57BL/6 mouse with the age of 6-8 weeks.

H&E staining materials: H&E staining materials: fixative, hematoxylin staining solution, eosin staining solution, dilute hydrochloric acid ethanol solution, culture flask, culture dish, ophthalmic forceps, coverslip, slide, microscope.

Immunohistochemical/fluorescence analysis materials: sections, reagents/kits, instruments/consumables.

### Experimental Methods

(1) Model construction: Homograft of living cells of pancreatic cancer tumor cell line PAN02 was implanted into C57BL/6 mouse subcutaneously. Volume testing and dissection analysis for different volumes were performed; tissue section H&E staining/collagen staining and immunohistochemical/fluorescence analysis were performed.
(2) Gradient administration of mutant collagenase and optimization of conditions: the inoculated model mouse was administrated; survival analysis and side effect analysis were performed; analysis of tumor volume, texture and metastasis was performed; tissue section H&E staining/collagen staining and immunohistochemical/fluorescence analysis were performed; flow cytometry analysis of tumor-infiltrating immune cells (TILS) was performed.

### Experimental Results

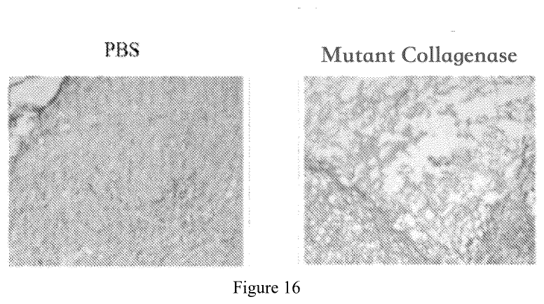

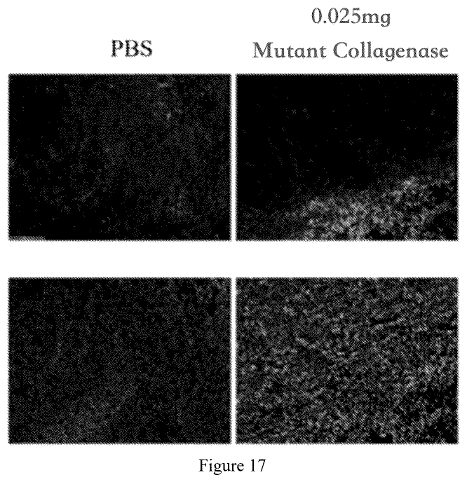

Injection of 2 points, 100 μl, 0.0125 mg, 300+100 mm³.
(1) The tumor collagen decreased, and the abundance decreased (with specific reference to FIG. 16).
(2) The tumor became soft, and the tissue became loose.
(3) The infiltration of immune cells was obvious (with specific reference to FIG. 17).

### Example 6 Injection of Mutant Collagenase for PAN02 Pancreatic Ductal Adenocarcinoma Mouse Model

### Instruments and Materials

PAN02 pancreatic cancer tumor cell line, twenty C57BL/6 mice with the age of 6-8 weeks.

### Experimental Methods

(1) Homograft of living cells of pancreatic cancer tumor cell line PAN02 was implanted into C57BL/6 mouse subcutaneously. Ten mice selected were injected with only PBS as control, ten mice were injected with only 0.0125 mg/100 μl mutant collagenase.
(2) Changes of tumor size were observed 45, 50, 55, 60 days after the injection.

### Experimental Results

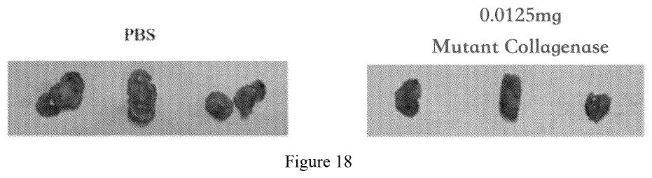

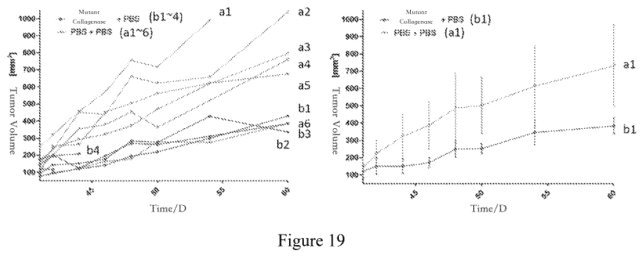

(1) Six mice in the control group survived, and four mice in the administrated group survived.
(2) Compared with the control group, the tumor volume in the group injected with mutant collagenase was significantly reduced (FIG. 18).
(3) Compared with the control group, the growth of the tumor of in the group injected with mutant collagenase showed a trend of tumor growth being inhibited (FIG. 19, Note: a1˜a6 are in the control group, b1˜b4 are in administrated group).

### REFERENCES

1. World Health Organization. WHO report on cancer: setting priorities, investing wisely and providing care for all. 2020.
2. Siegel R L, Miller K D, Jemal A. Cancer statistics, 2020. CA: A Cancer Journal for Clinicians. 2020;70 (1): 7-30.
3. World Health Organization. <13-Pancreas-fact-sheet.pdf>. 2020.
4. World Health Organization. <16-Melanoma-of-skin-fact-sheet.pdf>. 2020.
5. Fang M, Yuan J, Peng C, Li Y. Collagen as a double-edged sword in tumor progression. Tumor Biology. 2014;35 (4): 2871-82.
6. Henke E, Nandigama R, Ergün S. Extracellular matrix in the tumor microenvironment and its impact on cancer therapy. Frontiers in Molecular Biosciences. 2020;6:160.
7. Riegler J, Labyed Y, Rosenzweig S, Javinal V, Castiglioni A, Dominguez C X, et al. Tumor elastography and its association with collagen and the tumor microenvironment. Clinical Cancer Research. 2018;24 (18):4455-67.
8. Dolor A, Szoka Jr F C. Digesting a path forward: the utility of collagenase tumor treatment for improved drug delivery. Molecular pharmaceutics. 2018;15 (6): 2069-83.
9. Han Y, Liu D, Li L. PD-1/PD-L1 pathway: current researches in cancer. American Journal of Cancer Research. 2020; 10 (3):727.
10. Wu Y, Chen W, Xu Z P G, Gu W. PD-L1 distribution and perspective for cancer immunotherapy-blockade, knockdown, or inhibition. Frontiers in Immunology. 2019;10:2022.
11. Goodman AM, Piccioni D, Kato S, Boichard A, Wang H-Y, Frampton G, et al. Prevalence of PDL1 amplification and preliminary response to immune checkpoint blockade in solid tumors. JAMA oncology. 2018;4 (9): 1237-44.
12. Imbert C, Montfort A, Fraisse M, Marcheteau E, Gilhodes J, Martin E, et al. Resistance of melanoma to immune checkpoint inhibitors is overcome by targeting the sphingosine kinase-1. Nature communications. 2020;11 (1): 1-14.
13. Lei Q, Wang D, Sun K, Wang L, Zhang Y. Resistance Mechanisms of Anti-PD1/PDL1 Therapy in Solid Tumors. Frontiers in Cell and Developmental Biology. 2020;8.

1. A method of cancer treatment, comprising: injecting a composition comprising mutant collagenase into a tumor of an individual with cancer to reduce the abundance of collagen in the extracellular matrix of the tumor microenvironment.

2. The method of claim 1, wherein said tumor is a solid tumor.

3. The method of claim 1, wherein the mutant collagenase in said composition is recombinant mutant collagenase with purity of more than 98, and the amino acid sequence of said recombinant mutant collagenase is given by SEQ ID NO: 2.

4. The method of claim 1, wherein said injecting is multi-point injection or two-point injection, and the concentration of said mutant collagenase in said composition is 0.005-0.15 mg/100 μl.

5. The method of claim 1, wherein said method is can be applied in combination with chemotherapy, immunotherapy or targeted therapy.

6. The use of a composition comprising mutant collagenase in the manufacture of a medicament for the treatment of cancer.

7. The use of claim 6, wherein said cancer is malignant tumor.

8. The use of claim 6, wherein the mutant collagenase in said composition is recombinant mutant collagenase with purity of more than 98%, and the amino acid sequence of said recombinant mutant collagenase is given by SEQ ID NO: 2.

9. The use of claim 6, wherein said composition further comprises a pharmaceutically acceptable carrier.

10. The use of claim 6, wherein the dosage form of said composition is injection.


SEQUENCE LISTING (2 sequences)

SEQ ID NO: 1 - DNA, 2946 bp, Artificial Sequence
  Sythetic
atggttcagaatgagagtaaacgttataccgttagctacctgaaaacactgaactattatgatctggtggatctgctggtgaagacagagattgaaaacttacctgatctgttccagtacagcagcgatgccaaagagttttacggcaacaagacccgtatgagcttcatcatggacgagattggccgtcgtgcccctcagtataccgaaatcgaccataagggcatcccgaccctggttgaggttgtgcgtgccggtttctacctgggcttccacaataaagagctgaacgaaatcaataaacgtagctttaaggagcgcgtgattcctagtattctggccattcagaaaaatcctaatttcaagctgggtactgaagtgcaggacaaaatcgttagcgccacggggttactggcaggcaatgaaaccgccccgccggaggttgtgaataacttcaccccgatcttgcaggactgcatcaaaaacatcgaccgctatgccctggacgacctgaaaagtaaggcactgttcaacgtgctggccgcacctacatacgatatcacagaatacttacgcgccaccaaggagaaaccggagaataccccgtggtacggcaagattgacggcttcattaacgagctgaaaaaactggcattatacggcaaaatcaatgacaacaacagctggattatcgataatggcatttaccacattgcacctctgggtaaactgcatagcaacaataaaattggtattgagaccctgaccgaagttatgaaagtttacccgtacctgagcatgcagcatctgcaaagcgccgatcaaatcaaacgccactacgatagcaaggatgccgaaggcaacaagatccctctggataaatttaaaaaagaaggtaaagaaaaatattgtccgaaaacctatacatttgatgatggcaaagttattattaaggcaggcgcacgcgtggaagaggagaaagtgaaacgcctgtattgggccagcaaagaggtgaacagtcagttcttccgcgtttatggcatcgacaagccgctggaagaaggcaacccggatgacattctgacgatggtgatctataacagcccggaggagtacaagctgaatagtgtgttatatggttatgataccaataatggtggtatgtatatcgagccggaaggcaccttcttcacctatgagcgtgaagcccaagagagtacctataccctggaggagctgttccgtcacgagtatacacactacctgcaaggccgctatgcagttccgggtcaatggggccgcaccaaactgtatgacaacgaccgtctgacctggtatgaggagggcggtgcagacttatttgccggtagtacccgtaccagcggtattctgccgcgtaagagcatcgttagcaacattcataataccacacgtaacaatcgttacaaactgagtgataccgttcacagcaaatacggcgcaagtttcgaattttataattacgcatgcatgtttatggactacatgtataataaagatatgggcatcctgaataaactgaatgatctggccaagaataatgatgttgacggttacgataactacatccgcgatctgagcagcaattacgccctgaatgataagtatcaggaccacatgcaggagcgcatcgacaattacgagaacttaaccgtgccgttcgttgccgacgactacctggttcgtcatgcctataagaatccgaatgaaatctacagtgaaattagcgaggttgcaaagctgaaagacgccaagagcgaggtgaaaaaaagtcagtatttcagtaccttcaccttacgcggcagttacacggggggcgccagcaagggtaagctggaagaccagaaagccatgaataaatttatcgatgatagcttaaaaaaattagatacctatagctggagtggctacaaaaccctgaccgcatacttcaccaactataaggtggatagcagtaatcgcgtgacctatgacgtggtgtttcacggctacctgccgaatgagggtgatagcaagaacagcttaccgtacggtaagatcaacggcacctacaagggcaccgaaaaggagaagattaagttcagcagtgaaggcagcttcgaccctgacggcaaaattgtgagttacgagtgggacttcggcgatggcaacaagagcaacgaggagaacccggaacacagttacgacaaggtgggcacctacacagtgaaactgaaagtgaccgatgacaaaggcgaaagcagcgttagcaccacaaccgcagagatcaaagacttaagcgagaataaactgccggtgatttacatgcacgtgccgaaaagtggcgccctgaaccagaaagtggtgttttatggcaaaggtacatacgacccggatggcagcatcgccggttatcagtgggattttggcgacggcagtgatttcagcagcgagcagaacccgagtcatgtttacaccaagaagggcgaatataccgtgaccctgcgtgtgatggacagcagcggccagatgagtgaaaaaaccatgaaaatcaaaattaccgacccggtgtacccgattggcaccgagaaagaaccgaacaacagcaaggagaccgccagcggccctatcgttcctggtattcctgttagcggcaccattgagaacacaagcgatcaggactatttctattttgatgtgatcaccccgggcgaagtgaagattgacattaacaaactgggttatggtggcgccacctgggtggtgtacgatgagaacaacaatgccgtgagttacgcaaccgacgatggccagaatctgagcggcaaattcaaagccgacaagccgggtcgctattacattcatctgtatatgttcaacggcagctacatgccgtatcgtattaacattgaaggtagcgtgggtcgc

SEQ ID NO: 2 - protein, 982 aa, Artificial Sequence
  Synthetic
MVQNESKRYTVSYLKTLNYYDLVDLLVKTEIENLPDLFQYSSDAKEFYGNKTRMSFIMDEIGRRAPQYTEIDHKGIPTLVEVVRAGFYLGFHNKELNEINKRSFKERVIPSILAIQKNPNFKLGTEVQDKIVSATGLLAGNETAPPEVVNNFTPILQDCIKNIDRYALDDLKSKALFNVLAAPTYDITEYLRATKEKPENTPWYGKIDGFINELKKLALYGKINDNNSWIIDNGIYHIAPLGKLHSNNKIGIETLTEVMKVYPYLSMQHLQSADQIKRHYDSKDAEGNKIPLDKFKKEGKEKYCPKTYTFDDGKVIIKAGARVEEEKVKRLYWASKEVNSQFFRVYGIDKPLEEGNPDDILTMVIYNSPEEYKLNSVLYGYDTNNGGMYIEPEGTFFTYEREAQESTYTLEELFRHEYTHYLQGRYAVPGQWGRTKLYDNDRLTWYEEGGADLFAGSTRTSGILPRKSIVSNIHNTTRNNRYKLSDTVHSKYGASFEFYNYACMFMDYMYNKDMGILNKLNDLAKNNDVDGYDNYIRDLSSNYALNDKYQDHMQERIDNYENLTVPFVADDYLVRHAYKNPNEIYSEISEVAKLKDAKSEVKKSQYFSTFTLRGSYTGGASKGKLEDQKAMNKFIDDSLKKLDTYSWSGYKTLTAYFTNYKVDSSNRVTYDVVFHGYLPNEGDSKNSLPYGKINGTYKGTEKEKIKFSSEGSFDPDGKIVSYEWDFGDGNKSNEENPEHSYDKVGTYTVKLKVTDDKGESSVSTTTAEIKDLSENKLPVIYMHVPKSGALNQKVVFYGKGTYDPDGSIAGYQWDFGDGSDFSSEQNPSHVYTKKGEYTVTLRVMDSSGQMSEKTMKIKITDPVYPIGTEKEPNNSKETASGPIVPGIPVSGTIENTSDQDYFYFDVITPGEVKIDINKLGYGGATWVVYDENNNAVSYATDDGQNLSGKFKADKPGRYYIHLYMFNGSYMPYRINIEGSVGR